(19) United States
(12) Patent Application Publication
(10) Pub. No.: US 2007/0255086 A1
(43) Pub. Date: Nov. 1, 2007

(54) Medical Device Including Magnetic Particles

(76) Inventor: Robert J. Nehls, Lakeville, MN (US)

Correspondence Address: MEDTRONIC, INC., 710 MEDTRONIC PARKWAY NE, MINNEAPOLIS, MN, 55432-9924, US

(21) Appl. No.: 11/380,225

(22) Filed: Apr. 26, 2006

(51) Int. Cl. A61N 2/00

(52) U.S. Cl. 600/12

(57) ABSTRACT

An implantable medical device includes a plurality of magnetic particles contained by a biodegradable material.

### TECHNICAL FIELD

The present invention pertains to implantable medical devices and more particularly to magnetically guided implantable medical devices.

### BACKGROUND

Magnetic navigation provides alternative methods for implanting medical devices, for example, electrical leads, therapeutic agent delivery tubes or seeds, and stents, within a body of a patient. Magnetic navigation manipulates a magnetic field, formed by a pair of magnets positioned external to the patient, to direct, or orient, an internal magnet of the device, thereby facilitating steering of the device to a target implant site. Such a system is provided by Stereotaxis and is described, for example, in U.S. Pat. No. 6,015,414. Alternately, an attractive force between an external magnet and the internal magnet provides enough traction force to move the device to the implant site, for example as described in U.S. Pat. Nos. 4,162,679 and 6,126,647 and 6,212,419.

With the advent of magnetic resonance imaging (MRI), it may not be desirable for an implanted device to include a magnet such as would be useful for magnetic navigation, since the relatively large magnetic field used in MRI may induce injury via interaction with the device's magnet. Thus, there is a need for magnetically navigable implantable device that is MRI compatible.

### BRIEF DESCRIPTION OF THE DRAWINGS

The following drawings are illustrative of particular embodiments of the present invention and therefore do not limit the scope of the invention. The drawings are not to scale (unless so stated) and are intended for use in conjunction with the explanations in the following detailed description. Embodiments of the present invention will hereinafter be described in conjunction with the appended drawings, wherein like numerals denote like elements.

FIG. 1 is a schematic showing an exemplary implanted device according to some embodiments of the present invention.

FIG. 2A is a plan view, including a partial section, of the device shown in FIG. 1, according to some embodiments of the present invention.

FIG. 2B is a detailed section view of a portion of a device, according to some alternate embodiments of the present invention.

FIG. 3A is a plan view, including a partial section, of an exemplary device, according to some embodiments of the present invention.

FIG. 3B is a detailed section view of a portion of the device shown in FIG. 3A, according to some alternate embodiments of the present invention.

FIG. 4 is a plan view of a device, according to additional embodiments of the present invention.

FIG. 5 is a section view of a device, according to yet another embodiment of the present invention.

### DETAILED DESCRIPTION

The following detailed description is exemplary in nature and is not intended to limit the scope, applicability, or configuration of the invention in any way. Rather, the following description provides practical illustrations for implementing exemplary embodiments of the present invention. Constructions, materials, dimensions, and manufacturing processes suitable for making embodiments of the present invention are known to those of skill in the field of the invention.

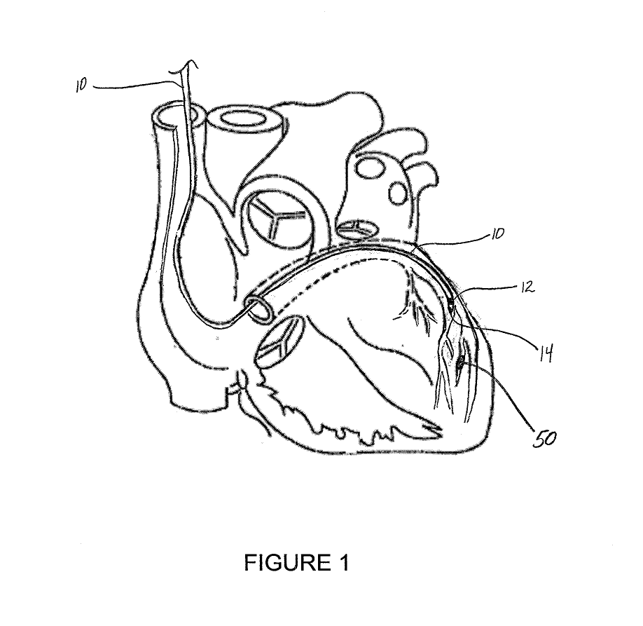

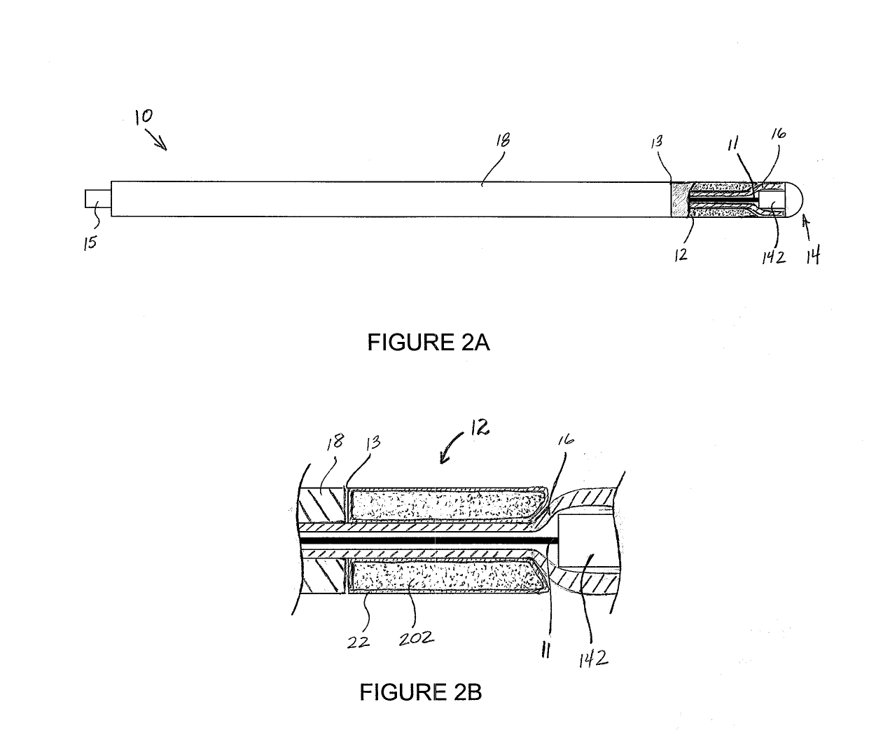

FIG. 1 is a schematic showing an exemplary implanted device 10 according to some embodiments of the present invention. FIG. 1 illustrates device 10 having been implanted within a coronary vein; device 10 includes a magnetic element 12 and a tip electrode 14. According to embodiments of the present invention, device 10 has been guided to the illustrated implant site using a magnetic attraction between an external magnet or magnetic field and magnetic element 12. FIG. 2A is a plan view, including a partial section, of device 10, according to some embodiments of the present invention. FIG. 2A illustrates magnetic element 12 formed as a coating or a sleeve extending about an insulation tube 16 through which a conductor 11 extends; conductor 11 is coupled to a proximal connector pin 15 and extends to electrode 14, being joined to a shank 142 of electrode 14, for example by crimping, in order to electrically couple electrode 14 to connector pin 15. FIG. 2A further illustrates device 10 including a body 18, which may augment the insulation of conductor 11 or may carry an additional conductor for another electrode (not shown), and element 12, which has an outer diameter approximately equal to that of body 18.

According to the illustrated embodiment, magnetic element 12 comprises a plurality of magnetic particles embedded within a matrix of a biocompatible and biodegradable material, examples of which include, but are not limited to, polysaccharides, such as mannitol, sorbitol and sucrose, synthetic polymers, such as polyglycolic acid (PGA), polyactic acid (PLA) and polyacticitide-co-glycolide (PLGA), and natural biopolymers, such as collagen and albumin. After magnetic element 12 has served its purpose in the implantation of device 10, the matrix material of magnetic element 12 will degrade, being in contact with bodily fluids, to release the plurality of magnetic particles into the bloodstream. According to an exemplary embodiment of the present invention, the plurality of magnetic particles comprise iron oxide nano-particles, and the particles are blended with the biocompatible and biodegradable material, according to methods known to those skilled in the art, such that element 12 contains a concentration of magnetic particles being between approximately 20% and approximately 90%, by weight, preferably, between 40% and 60%. According to some embodiments of the present invention, each particle of the plurality of magnetic particles includes a biologically inert and passivating nano-coating, for example, alumina, as described by George et al. in U.S. Pat. No. 6,913,827, salient portions of which are hereby incorporated by reference. Other magnetic materials which may be employed by the present invention include, but are not limited to, cobalt, boron, zinc, nickel, zinc manganese, cadmium, barium and alloys thereof.

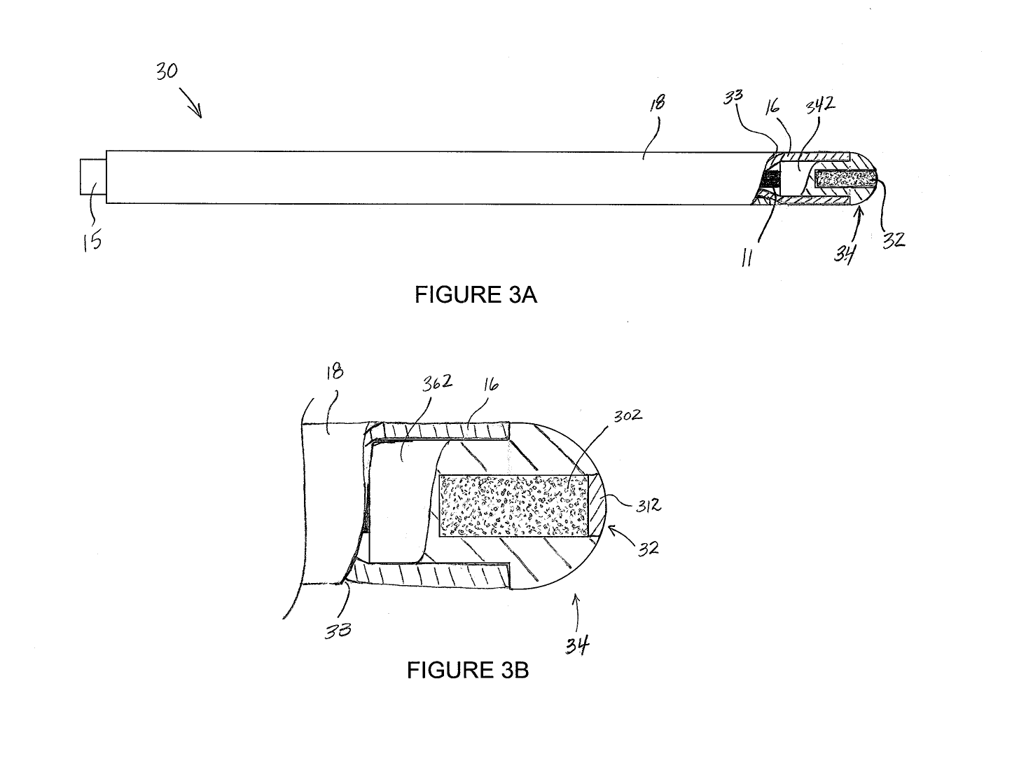

FIG. 3A is a plan view, including a partial section, of an exemplary device 30, according to some embodiments of the present invention. FIG. 3A illustrates a magnetic element 32, similar to embodiments of element 12, previously described, in that element 32 comprises a plurality of magnetic particles contained within a matrix formed by a biocompatible and biodegradable material. In contrast to element 12, element 32 is shown formed as a plug held within a bore of an electrode tip 34; the bore of electrode 34 is open at a distal end allowing exposure of the matrix containing the plurality of magnetic particles to degrading bodily fluids.

FIGS. 2B and 3B are section views of portions of devices 10 and 30, respectively, showing alternate embodiments of elements 12 and 32. FIG. 2B illustrates element 12 including a plurality of magnetic particles 202 contained within sidewalls of a shell or housing 22, which is formed from a biocompatible and biodegradable material. FIG. 3B illustrates element 32 including a plurality of magnetic particles 302 contained within a housing formed by the bore of electrode 34 and a cap 312, which is formed from a biocompatible and biodegradable material. According to an exemplary embodiment of the present invention, particles 202 and 302 are a blend of iron and carbon powders, for example, as described by Volkonsky et al. in U.S. Pat. No. 6,200,547, salient portions of which are hereby incorporated by reference. Alternately, particles 202 and 302 may be any of those previously described in conjunction with FIGS. 2A and 3A.

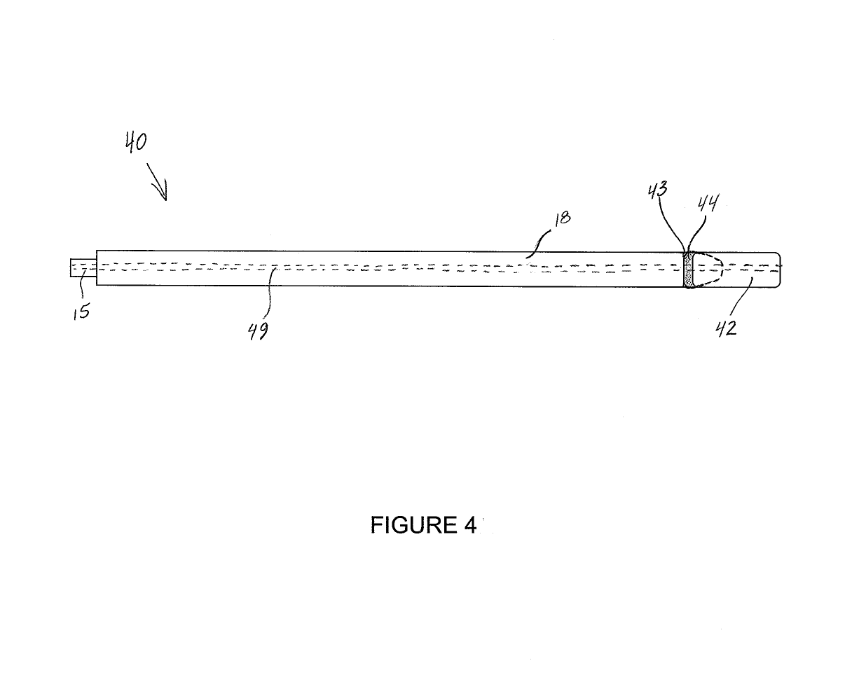

FIG. 4 is a plan view of a device 40, according to additional embodiments of the present invention. FIG. 4 illustrates device 40 including a distal electrode 44, which is partially encapsulated by a magnetic element 42 that forms a distal tip of device 40. Element 42 includes a plurality of magnetic particles either contained within a matrix of biodegradable material, for example, as described in conjunction with FIGS. 2A and 3A, or contained within a sidewall of a housing formed, at least in part, by a biodegradable material, for example, as described in conjunction with FIGS. 2B and 3B. FIG. 4 further illustrates device 40 including a lumen 49, shown with dashed lines, which may extend within a conductor coil (not shown) coupling electrode 44 to electrode pin 15, or within a separate lumen of body 18, alongside a cable conductor, for example, conductor 11 shown in FIGS. 2A-3B. Lumen 49 may provide a passageway for delivery of therapeutic agents or other devices.

Referring now to FIGS. 2A-4, according to certain embodiments of the present invention, body 18 of devices 10, 30, and 40 has a diameter between approximately 0.025 inch and approximately 0.055 inch, and, being relatively supple, or flexible, may not have the stiffness necessary to apply the necessary push force to implant electrode 14, 34, 44, for example, at or near the site at which electrode 14 is shown in FIG. 1. According to these embodiments, a magnetic traction applied by an external permanent magnet, or an electromagnet, may be used to pull devices 10, 30 and 40, via elements 12, 32 and 42, respectively. Alternately, if lead bodies 18 can support a push force from proximal end, a relatively smaller magnetic field may simply move elements 12, 32 and 42 with respect to lead bodies 18, for example, via flexure at junctions 13, 33 and 43, respectively, to guide or direct the tip electrodes 14, 34 and 44, as an aid in steering device 10, 30 to the implant site.

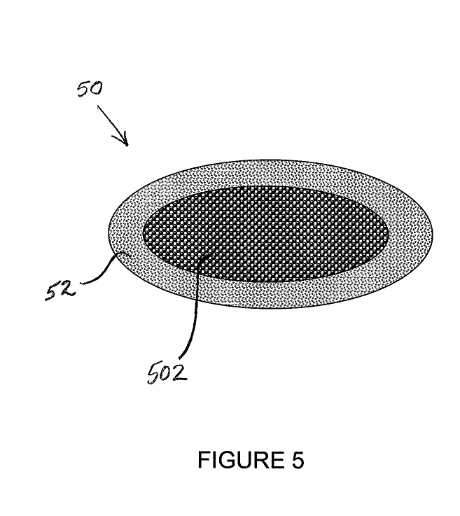

FIG. 5 is a section view of a device 50, according to yet another embodiment of the present invention. FIG. 5 illustrates device 50 formed as a pellet or seed that includes a matrix of biocompatible and biodegradable material 52 containing a therapeutic agent, for example, an anti-inflammatory, anti-fungal, anti-tumor or chemotherapy agent; matrix 52 forms a shell to further contain a plurality of magnetic particles 502 therein. The material of matrix 52 may be any of the materials previously described, and magnetic particles 502 may be any of those previously described. According to embodiments of the present invention, seed 50 is directed, within a body of a patient, to an implant site via an external magnetic field, for example as described by Blume et al. in U.S. Pat. No. 6,212,419, salient portions of which are hereby incorporated by reference. According to some methods of the present invention seed 50 may be directed to an implant site wherein seed 50 is lodged, for example, within brain tissue of the patient, or within a relatively small vessel of the circulatory system of the patient, for example, as illustrated in FIG. 1. Once lodged, matrix 52 will completely degrade, over time, first releasing the therapeutic agent to the target site, and then releasing magnetic particles 502 to disperse away from the site. Alternately, the external magnetic field may be applied to hold pellet 50 at a target site (for example, if the site is not sufficiently small for pellet 50 to become lodged therein) over a period of time in which matrix 52 degrades to release the agent. According to an alternate embodiment, matrix 52 may extend throughout pellet 50 to contain both the therapeutic agent and particles 502 by embedment therein.

In the foregoing detailed description, the invention has been described with reference to specific embodiments. However, it may be appreciated that various modifications and changes can be made without departing from the scope of the invention as set forth in the appended claims. For example, the scope of the invention covers any implantable device including a solid biodegradable structure containing magnetic particles or a structural portion to which a biodegradable magnetic element may be coupled. Examples of these other devices, include, but are not limited to indwelling catheters, or delivery tubes, and stents, which are well known to those skilled in the art.

1. A medical electrical lead, comprising a distal end and a plurality of magnetic particles contained within a matrix of biodegradable material, the matrix disposed in proximity to the distal end of the lead.

2. The lead of claim 1, further comprising an insulated conductor and wherein the matrix surrounds a portion of the insulated conductor.

3. The lead of claim 2, further comprising a tip electrode coupled to the conductor, the matrix being disposed adjacent the tip electrode.

4. The lead of claim 2, further comprising a body through which the insulated conductor extends, the matrix being approximately concentric with the body and having an outer diameter approximately equal to an outer diameter of the body, and the outer diameter of the body being between approximately 0.025 inch and 0.055 inch.

5. The lead of claim 1, further comprising a tip electrode including a bore extending therein and being in fluid communication with an exterior environment of the lead, the matrix being disposed within the bore.

6. The lead of claim 1, further comprising a tip electrode and wherein the matrix at least partially encapsulates the tip electrode.

7. The lead of claim 1, wherein the matrix forms a distal tip of the lead.

8. The lead of claim 1, wherein each of the plurality of magnetic particles includes a biologically inert coating.

9. A medical electrical lead, comprising a plurality of magnetic particles enclosed within a housing, the housing being, at least in part, biodegradable.

10. The lead of claim 9, further comprising an insulated conductor and wherein the housing surrounds a portion of the insulated conductor.

11. The lead of claim 10, further comprising a tip electrode coupled to the conductor, the housing being disposed adjacent the tip electrode.

12. The lead of claim 10, further comprising a body through which the insulated conductor extends, the housing being approximately concentric with the body and having an outer diameter approximately equal to an outer diameter of the body, and the outer diameter of the body being between approximately 0.025 inch and 0.055 inch.

13. The lead of claim 9, further comprising a tip electrode including a bore extending therethrough, the housing being formed by the bore and a biodegradable cap separating the bore from an environment external to the lead.

14. The lead of claim 9, wherein each of the magnetic particles includes a biologically inert coating.

15. An implantable medical device, comprising a plurality of magnetic particles contained by a biodegradable material.

16. The device of claim 15, wherein the biodegradable material forms a solid.

17. The device of claim 16, further comprising a therapeutic agent contained by the biodegradable material.

18. The device of claim 15, wherein the biodegradable material forms a matrix, the plurality of magnetic particles being embedded within the matrix.

19. The device of claim 15, wherein the biodegradable material forms at least part of a housing containing the plurality of magnetic particles.

20. The device of claim 15, wherein each of the plurality of magnetic particles includes a biologically inert coating.
